(19) United States
(12) Patent Application Publication
(10) Pub. No.: US 2012/0121519 A1
(43) Pub. Date: May 17, 2012

(54) NATURAL POLYMERIC EMULSIFIERS AND METHOD FOR USE

(75) Inventors: John S. THOMAIDES, Berkeley Heights, NJ (US); Joseph B. GARDNER, Lexington, OH (US); Qiwei HE, Belle Mead, NJ (US); Robin C. PENNELL, Riegelsville, PA (US)

(73) Assignee: AKZO NOBEL CHEMICALS INTERNATIONAL B.V., Amersfoort (NL)

(21) Appl. No.: 13/290,611

(22) Filed: Nov. 7, 2011

Related U.S. Application Data
(60) Provisional application No. 61/412,542, filed on Nov. 11, 2010.

(30) Foreign Application Priority Data
Mar. 31, 2011 (EP) 11160686.9

(51) Int. Cl. A61K 8/06; C08B 31/16; A61Q 17/04; A61Q 5/06; A61Q 5/12; C08B 31/08; A61K 8/92

(52) U.S. Cl. 424/43; 536/47; 424/59; 424/70.13

(57) ABSTRACT

Polymeric emulsifiers include polysaccharides modified with at least one cross-linking reagent and with from about 1 mol % to about 10 mol % of at least one ionic reagent, methods for preparing the same, and emulsions including the polymeric emulsifiers.

### CROSS REFERENCE TO RELATED APPLICATION

This application claims the benefit of priority to U.S. Provisional Patent Application Ser. No. 61/412,542, filed Nov. 11, 2010 and EP Application No. 11160686.9, filed Mar. 31, 2011, each of which is incorporated by reference in its entirety herein.

### FIELD OF INVENTION

The present invention relates to polymeric emulsifiers. More specifically, the present invention relates to polymeric emulsifiers derived from natural materials, such as starch.

### BACKGROUND OF THE INVENTION

Starches modified with substituted succinic anhydride reagents have been used for making emulsions for food and industrial applications. The most common of these is octenyl succinic anhydride (OSA), which has worked well in food systems, particularly for emulsifying flavor oils.

OSA modified starches have also been used in cosmetic areas. However, conventional OSA modified starches do not provide sufficient emulsion stability for many cosmetic applications where more hydrophobic oils are applied to the skin or hair, or they have to be present in large amounts relative to the emulsified oil in order to work. Emulsion stability is also important as long term shelf storage is needed for commercial emulsions. Examples of such emulsions are sunscreens, skin moisturizing formulations, and skin creams.

In these cosmetic formulations, synthetic materials have typically been used as emulsifiers. However, small molecule emulsifiers have led to irritation, toxicity, and negative interactions with the cosmetic functional materials in the formulations. In view of the above problems, there is a need to develop emulsifiers that can provide long term emulsion stability, as well as exhibit improved biodegradability and sustainability.

### SUMMARY OF THE INVENTION

Accordingly, the present invention relates to polymeric emulsifiers having improved biodegradability and sustainability as well as exemplary emulsification loading and long term storage stability that can either eliminate the need for added small molecule emulsifiers or greatly reduce the amount of small molecule emulsifiers needed to achieve a stable emulsion. It has been surprisingly found that a starch that has been cross-linked at a certain level, functionalized with the proper amount of ionic reagent, and gelatinized can act as an efficient emulsifier for oil-in-water emulsions.

In one aspect, the invention relates to an emulsion. The emulsion comprises a polymeric emulsifier comprising a polysaccharide. The polysaccharide is modified with at least one cross-linking reagent and with from about 1 mol % to about 10 mol % of at least one ionic reagent. The emulsion further includes a cosmetically acceptable oil and a solvent system. The solvent system is in the continuous phase.

In another aspect, the invention is directed to a polymeric emulsifier comprising a polysaccharide. The polysaccharide is modified with at least one cross-linking reagent and with from about 1 mol % to about 10 mol % of at least one cationic reagent. The polysaccharide is further modified with at least about 15 ppm and less than 100 ppm of the cross-linking reagent, based on the weight of the ionically modified polysaccharide, such as starch. In another aspect, the invention relates to a polymeric emulsifier comprising a polysaccharide. The polysaccharide is modified with at least one cross-linking reagent and with from about 1 mol % to about 10 mol % of at least one anionic containing reagent. The polysaccharide is further modified with from about 15 ppm to about 375 ppm of the cross-linking reagent, based on the weight of the ionically modified starch.

### DETAILED DESCRIPTION OF THE INVENTION

The present invention relates to polymeric emulsifiers exhibiting improved biodegradability and sustainability as well as suitable emulsification loading and long term emulsion stability. Generally, an emulsion comprises a mixture of two immiscible substances, one substance (the dispersed phase) dispersed in the other (the continuous phase). In an aspect of the invention, a polymeric emulsifier comprises a starch modified with a cross-linking reagent and an ionic reagent. These polymeric emulsifiers can be used to form emulsions including a cosmetically acceptable oil dispersed in water. For the purposes of this invention, an emulsion is defined as a plurality of oil droplets substantially uniformly distributed or dispersed in a solvent system. The solvent system forms the continuous phase, and the oil is not soluble in the solvent system. Some non-limiting examples of suitable solvent systems include water, ethanol, methanol, isopropanol, glycerol, propylene glycol, or acetone or mixtures thereof. In an embodiment of the invention, the solvent is a mixture of water with one or more of ethanol, methanol, isopropanol, glycerol, propylene glycol, or acetone.

In an embodiment of the invention, the plurality of oil droplets in the emulsion have a mean average particle size of from about 0.2 microns to about 100 microns. In another embodiment, the oil droplets have a mean average particle size of from about 0.5 microns to 35 microns. In yet another embodiment, the oil droplets have a mean average particle size of from about 1 micron to about 25 microns. In embodiments of the invention, the mean average particle size can have a lower limits of 0.2 microns, 0.5 microns and 1 micron, respectively, while the upper limits can be 100 microns, 35 microns and 25 microns, respectively, with embodiments having ranges being combinations of these lower and upper limits

In an embodiment of the invention, the cosmetically acceptable oil is an oil that is not soluble in the solvent system and provides some benefit such as feel, protection, healing, UV protection, occlusion, slip, hydration, or radical scavenging to the consumer. Non-limiting examples of cosmetically acceptable oils are mineral oil, petroleum jelly, petrolatum, silicone, dimethicone, emu oils, castor oils, squaline, avocado oil, almond oil, coconut oil, cocoa butter, grapeseed oil, lanolin, peanut oil, sesame oil, jojoba oil, olive oil, shea butter, and wheat germ oil.

In an embodiment of the invention, the cosmetically acceptable oil may be an aerosol propellant.

In an embodiment of the invention, the polymeric emulsifier of the present invention is a naturally derived polymeric emulsifier wherein the emulsifier is formed from starch. The starch material of this invention can be isolated from any plant source of starch, including, for example, corn, wheat, rice, sorghum, pea, potato, tapioca (cassava), sweet potato, and sago. In an embodiment of this invention, the starch contains greater than about 90 percent of amylopectin. In another embodiment, the starch contains greater than 95 percent of amylopectin. In yet another embodiment, the starch contains greater than 97 percent of amylopectin. This high amylopectin starch is traditionally known in the art as waxy and there are many varieties of waxy starch commercially available. In an embodiment of this invention the waxy starch is of the corn, rice, potato, or tapioca. In an embodiment of the invention, the starch is gelatinized starch. In another embodiment, the gelatinized starch is in powder form.

In an embodiment of the invention, the starches will have a high molecular weight. For purposes of this invention, high molecular weight is defined as naturally occurring starches which have not been purposefully degraded to a lower molecular weight. That is, while some degradation may occur during the isolation of the starch and also during the chemical processing and drying of the starch, for purposes of the present invention, the high molecular weight starches are those that have their natural molecular weight maintained as much as possible. Measurement of molecular weight can be performed by a variety of techniques. For purposes of this invention, molecular weight values reported are those as would be determined by light scattering.

In some applications, such as where high solids levels are desirable, slight degradation of the starches can be performed. In an embodiment of the invention, the starches will have a weight average molecular weight of about 1 million or greater. In another embodiment, the starches have a weight average molecular weight of about 10 million or greater. In yet another embodiment, the starches have a weight average molecular weight of about 50 million or greater.

In an embodiment, the polymeric emulsifier is a starch that is modified with at least one first reagent by which the starch is cross-linked The at least one first reagent is thus a cross-linking reagent. Suitable cross-linking agents for use in the present invention are compounds that are capable of reacting with two or more hydroxyl groups found on the starch. Examples of such starch cross-linking reagents include epichlorohydrin epichlorohydrin, phosphorous oxychloride, sodium tri-meta phosphate (STMP), adipic/acetic anhydride, formaldehyde, adipoyl chloride, glyoxal and combinations thereof. In an embodiment of this invention, the cross-linking reagent is epichlorohydrin. In another embodiment of this invention the cross-linking reagent is STMP or phosphorus oxychloride. In an embodiment of the invention, the starch may be modified with from about 15 ppm to about 100 ppm of the cross-linking reagent. In another embodiment, the starch may be modified with from about 15 ppm to about 375 ppm of cross-linking reagent, and in yet another embodiment with from about 25 ppm to 75 ppm of the cross-linking reagent. In embodiments of the invention, the starch may be modified with a lower limit of about 15 ppm and 25 ppm, respectively, while the upper limits may be 375 ppm, 100 ppm and 75 ppm, respectively, with embodiments also having ranges being combinations of these lower and upper limits.

In an embodiment of the invention, the cationic starch may be modified with from about 15 ppm to about 100 ppm of the cross-linking reagent, and in another embodiment of from about 25 ppm to 100 ppm.

In still yet another embodiment, the anionic starch may be modified with from about 15 ppm to about 375 ppm of cross-linking reagent, and in another embodiment of from 15 ppm to about 100 ppm and in a further embodiment of from about 25 ppm to about 75 ppm.

In another embodiment, non-chemical methods of cross-linking, such as heat treatment of starch, are also possible for formulations where an all natural label is desirable. In an embodiment of the invention, the polymeric emulsifier comprises starch is also modified with at least one ionic reagent. The ionic reagents may be cationic, anionic, amphoteric or zwitterionic. For the purposes of the invention, a reagent that has a net anionic charge under testing conditions is referred to as “anionic” and thus is an anionic containing reagent. In an embodiment of the invention, the starch is modified with from about 1 mole % to about 10 mole % of the ionic reagent. In another embodiment, the starch is modified with from about 2 mole % to about 8 mole % and in yet another embodiment with from about 4 mole % to about 7 mole %. In embodiments of the invention, the starch is modified with at least about 1 mole %, at least 2 mole % and at least 4 mole %, respectively, and up to about 10 mole %, up to about 8 mole % and up to about 7 mole %, respectively, with embodiments having ranges being combinations of these lower and upper limits.

In an embodiment of the invention, the ionic reagent is a short chain cationic reagent, for example as shown in Structure 1 below. For purposes of this invention, “short chain cationic reagents” contain only C1 to C3 alkyl chains on the cationic center. The groups R, R′ and R″ can be the same alkyl group or they can be different alkyl groups or any combination thereof. In an embodiment of this invention R, R′ and R″ are methyl and X is chloride. In an embodiment, Y can be fluorine, chlorine, bromine, iodine or sulfate. Some non-limiting examples of short chain cationic reagents include 3-chloro-2-hydroxypropyltrimethyl ammonium chloride, 2,3-epoxypropyltrimethyl ammonium chloride, 2, 3-epoxypropyltriethyl ammonium chloride, 2-diethylaminoethyl chloride, and the like.

The cationic reagents of the polymeric emulsifier may also include long chain cationic reagents. In an embodiment of the invention, the long chain cationic reagents are used in combination with the short chain cationic reagents. Suitable long chain cationic reagents can include long chain quaternary amines that having a similar structure to the short chain cationic reagents of Structure 1, where R′ and R″ are alkyl groups having 1 to 3 carbon atoms and R contains alkyl or alkenyl groups having 8 to 18 carbon atoms. In another embodiment of this invention the R group will be a 12 carbon linear alkyl group and R′ and R″ will be methyl, as shown in Structure 1, below.

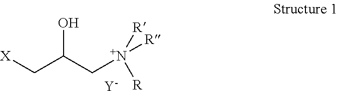

In an embodiment of the invention, R will be an alkyl group with from about 12 to about 18 carbon atoms. In an embodiment, X and Y each are chlorine.

One of ordinary skill in the art will recognize that the chlorohydrin can easily be converted to its epoxide form and still maintain the functional and reactive properties, both the epoxide and chlorohydrin form being reactive with starch hydroxyls. In an embodiment of the invention, the starch is further modified with from about 1 mole percent to about 5 mole percent of a long chain cationic reagent. In another embodiment, the starch is further modified with from about 0.1 mole percent to about 5 mole percent of a long chain cationic reagent and in yet another embodiment from about 1 mole percent to about 3 mole percent of a long chain cationic reagent. In embodiments of the invention, the starch is modified with at least about 0.1 mole percent, at least about 1 mole percent, respectively, as the lower limit and up to about 3 mole percent and at least about 5 mole percent, respectively, as the upper limit with embodiments having ranges being combinations of these lower and upper limits.

Alternatively, the starches of the current invention can be modified by complexation of a cross-linked starch with long chain quaternary material. Some non-limiting examples of long chain quaternary materials are octadecyl trimethylammonium chloride (ODAC), octadecyl triethylammonium chloride, lauryl trimethyl ammonium chloride, dodecyl ammonium chloride and the like.

In yet another embodiment, the polymeric emulsifier of the present invention can also be prepared from a cross-linked starch wherein the starch is modified by an anionic containing reagent in place of the cationic reagent. Examples of anionic containing reagents are amphoteric and anionic reagents, examples of which are CSPA (3-chloro-2-sulfopropionic acid) shown in Structure 2 and CEPA (2-chloroethylaminodipropionic acid) as shown in Structure 3.

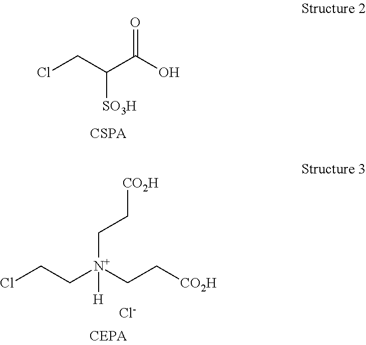

In an embodiment of the invention, the modification is with CEPA.

In the embodiment where an anionic containing modification and cross-linked starch is utilized as the polymeric emulsifier a minor amount of a non-ionic surfactant (non-polymeric emulsifier) may be added to increase or enhance stability. Examples of suitable emulsifiers are sorbitan esters, sorbitan oleate (SPAN 80), polyoxyethylene (20) sorbitan monooleate, and ethoxylates of long chain alcohols. In an embodiment of this invention, the polymeric emulsifier comprises an anionic polymeric emulsifier used in conjunction with SPAN or TWEEN surfactants.

The ability of the polymeric emulsifies of the present invention to form emulsions with very little or no added surfactants is one of the advantages of the present invention. This property allows for the production of emulsions that have very low toxicity or irritation to skin. The low irritation is desirable for finished formulations in the personal care industry, such as sunscreen formulations, skin lotions, skin creams, mousses, wipes, deodorants, antifungal creams, emollient lotions and creams, skin whitening emulsions, external self-tanning lotions and creams, skin brightening, hair relaxers, treatments for chapped skin, treatments for irritated skin, moisturizers such as creams and lotions, acne treatments, anti-wrinkle formulations, hair conditioners, hair mousses, styling gels, and shampoos.

In another aspect, the invention provides a method for making the polymeric emulsifier. The method comprises modifying a starch with at least one cross-linking reagent and at least one ionic reagent. The starch is modified with from about 15 ppm to about 100 ppm of the cross-linking reagent. In a further step, the starch is further modified with from about 1 to about 10 mole % of the cationic reagent.

Modification of the starch can be accomplished in any manner known in the art including alkaline aqueous reaction conditions in which the starch is modified in the granular state. For a general method for starch modification see “Modified starches: Properties and Uses”, O. B.Wurzburg, CRC Press, 1986, Boca Raton, Fla. See chapter 3 for cross-linking, chapter 8 for cationic derivatives, chapter 1 for gelatinization. Other methods for modification of starches are, for example, reactive extrusion, dry thermal reactions and solvent reactions. In one embodiment of this invention, the starches will be modified by aqueous alkaline reactions.

In modifying the starch with the at least one cross-linking reagent and the ionic reagent, the order of the modification reaction may depend on the reagents selected.

For example, if epichlorohydrin is the crosslinking agent and cationic 3-chloro-2-hydroxypropryl trimethylammonium chloride (QUAB® 188) is the ionic reagent, then the modification can be performed in any order, as both derivatives are attached by ether linkages. If either of the reagents is connected to the starch via a partially labile linkage (such as an ester) the reaction should be accomplished such that the last reagent provides the labile linkage. One skilled in the art of organic synthesis would recognize which reagents disclosed in this application form potentially labile linkages.

The modified granular starch is then gelatinized by cooking in water above the gelatinization temperature. Some non-limiting examples of gelatinization are bath cooking, steam injection cooking, jet cooking (at pressures of about 10 to about 150 PSI) and extrusion. It is believed that by gelatinizing the granular starch, the functionality as an emulsifier is obtainable. The starch of this invention can be cooked at a variety of temperatures and concentrations to provide the functional colloidal suspension. In an embodiment of this invention, the starch is cooked at about 90° C. to about 200° C. In another embodiment, the starch is cooked at about 100° C. to about 150° C. Depending on the method of cooking, limitations on the concentration of starch in water will vary due to factors, for example, such as viscosity, heat transfer and solution stability. In an embodiment of this invention, the starch will be cooked at concentrations from about 1% to about 40%; in another embodiment, the starch will be cooked at concentrations from about 2% to about 30%; and in yet another embodiment from about 3% to about 15%, or yet still other embodiments that are defined by combinations of the upper and lower limits of these ranges.

Once the starch is gelatinized in water the starch may be used without further manipulation or it can be recovered as the dry powder. Some illustrative examples of these processes are freeze drying, spray drying or precipitation into a polar, water miscible solvent (such as ethanol, isopropanol or acetone). This powder form offers the advantage of microbial stability (will not mold or mildew) and can be readily reconstituted into water with mixing. Some processes known in the art can be used to both gelatinize and recover the gelatinized dry powder. Examples of this class of processes include steam injected spray drying, drum drying and coupled jet cooking/spray drying. If the starch is not dried and stored as a solution in water, small amounts of one or more of the cosmetically acceptable preservatives that are known to those skilled in the art can be added to prevent the growth of microbes and mold. Preservatives can be selected from among methylparaben, propylparaben, butylparaben, DMDM hydantoin, imidazolidinyl urea, gluteraldehyde, phenoxyethanol, benzalkonium chloride, methane ammonium chloride, benzethonium chloride, benzyl alcohol, chlorobenzyl alcohol, methylchloroisothiazolinone, methylisothiazolinone, sodium benzoate, chloracetamide, triclosan, iodopropynyl butylcarbamate, sodium pyrithione, and zinc pyrithione.

In another aspect, the invention provides an emulsion. The emulsion comprises a polymeric emulsifier comprising a natural polymer modified with at least one cross-linking reagent and from about 1 mole % to about 10 mole % of at least one ionic reagent; a cosmetically acceptable oil; and water, wherein water is the continuous phase. The cosmetically acceptable oils used in the preparation of the emulsion have limited water solubility. In an embodiment of this invention, the oil forming the non-continuous phase will have a water solubility of about 1% or less, according to the Traditional Stability test described in the Examples section. In an embodiment of the invention, examples that could be prepared into an emulsion according to this invention include palm oil, mineral oil, silicone oil, sunflower oil, safflower oil, petrolatum and the like.

The products of this invention can also be used for a variety of industries and applications. Examples of such industries include as an aid in oil well drilling, laundry applications, crop protection, agriculture preparations, asphalt stabilizer, or coating aid.

Various other additives and active and functional ingredients may be included in the cosmetic composition as defined herein. These include, but are not limited to, emollients, humectants, thickening agents, surfactants, UV light inhibitors, fixative polymers, preservatives, pigments, dyes, colorants, alpha hydroxy acids, aesthetic enhancers such as starch, perfumes and fragrances, film formers (water proofing agents), antiseptics, antifungal, antimicrobial and other medicaments and solvents. Additionally, the cationic polygalactomannan or guar gum as found in this invention may be used in blends with other conditioning polymers and conditioning agents such as cationic hydroxyethyl cellulose, cationic synthetic polymers and cationic fatty acid derivatives. These blended materials help to provide more substantivity and effective conditioning properties in hair.

Surfactants which are useful in this invention include non-ionic and amphoteric surfactants. Non-ionic surfactants which may be used include polyoxyethyleneated, polyoxypropyleneated or polyglycerolated alcohols, alkylphenols and fatty acids with a linear fatty chain containing 8 to 18 carbon atoms and usually 2 to 30 mols of ethylene oxide, fatty acid amides, alkoxylated fatty alcohol alcohol amines, fatty acid esters, glycerol esters, alkoxylated fatty acid esters, sorbitan esters, alkoxylated sorbitan esters, alkylphenol alkoxylates, aromatic alkoxylates and alcohol alkoxylates. Also useful are copolymers of ethylene oxide and propylene oxide, condensates of ethylene oxide and propylene oxide with fatty alcohols, polyoxyethyleneated fatty amides or amines, ethanolamides, fatty acid esters of glycol, oxyethyleneated or non-oxyethyleneated fatty acid esters of sorbitan, fatty acid esters of sucrose, fatty acid esters of polyethylene glycols, phosphoric acid triesters and fatty acid esters of glucose derivatives.

The present invention will now be illustrated by the following non-limiting examples.

### EXAMPLES

### Preparation of Cross-Linked Waxy Maize Starch

In a 1 gallon plastic (Nalgene®) bottle, 1,345.84 g (7.4104 mole anhydroglucose units (AGUs)] waxy maize starch (National Starch, 89.2% solids) was slurried in deionized water using an overhead mechanical stirrer. To the stirred starch slurry was then added 323 mL (0.242 mole, 9.68 g) 3% (0.75 N) NaOH at a rate of 7.5 mL/minute using an automated dispenser. Target level of caustic was 0.8 wt. % vs. starch (100% solids basis). After the caustic addition was complete, the starch slurry was allowed to stir for 0.5 h. To the starch slurry was then added 200.0 □l (0.2366 g, 2.557 mmole) epichlorohydrin drop-wise over 5 minutes via syringe.

The bottle was then capped and placed in a heated (hot air) tumbler reactor set at 40° C. and allowed to tumble for 16 h. at 40° C. After 16 h at 40° C., the heater was turned off, and the reaction bottle was allowed to cool to room temperature while continuing to tumble.

The bottle was removed from the tumbler, and the pH of the reaction mixture was reduced to 5.1 from 11.9 by the drop-wise addition of concentrated HCl. The starch was isolated by vacuum filtration and then the filter cake was washed with 3×2 L deionized water on the funnel allowing the water to nearly entirely disappear into the filter cake between each wash (great care was taken to keep the filter cake from cracking). The product was then air dried to constant weight. The dried product thus obtained was ground to a powder using a coffee grinder. The yield of dried starch was 1,379.5 g (85.99% solids by moisture balance).

### Analysis of Cross-Linked Starch Using Settling Volume Test (SVT)

A Settling Volume Test (SVT) was run according the procedure of Rutenberg, et al. (Rutenberg, M. W.; Jarowenko, W.; Tessler, M. M. U.S. Pat. No. 4,048,435) to determine the extent of cross-linking. In summary: 1.00 g of the starch (100% solids basis) was taken up in about 95 g deionized water; the resulting slurry cooked in a boiling water bath for 20 minutes; the starch cook was made up to 100 g by the addition of the necessary amount of deionized water; the starch cook was vigorously stirred to homogenize it; the starch cook was poured into a 100 mL graduated cylinder, which was then sealed with aluminum foil; the cook was allowed to stand for 24 h at room temperature; and the volume of sediment vs. the total volume was measured. The Settling Volume (SV) is calculated by dividing the volume of sediment by the total volume and multiplying by 100 mL. The SV of this starch was determined to be 30.1 mL.

### Preparation of a Cationic Starch

A 1 L beaker was equipped with a mechanical stirrer and thermometer. To the beaker were charged 387.50 g deionized water and 50.0 g anhydrous sodium sulfate. After the sodium sulfate had completely dissolved, 289.75 g (1.542 mole AGU) of the cross-linked starch (86.28% solids) was charged to the reaction mixture. To the resulting slurry was added 66.00 mL (0.0502 mole, 2.0 g) of 3.03% (0.76 N) NaOH at a rate of 7.5 mL/minute using an automated dispenser. The pH of the reaction mixture was measured at this point and found to be 11.3 at 22° C. The temperature of the reaction mixture was then raised to 43° C. by immersing the beaker in a propylene glycol bath kept at 44° C. After about 0.5 hour at this temperature, the pH of the reaction mixture was measured and found to be 10.8 at 43° C.

In a separate beaker was charged 32.076 g (0.116 mole) of 3-chloro-2-hydroxypropyltrimethylammonium chloride (67.87% actives in water). This was cooled in an ice water bath. In a second separate beaker was charged 23.4935 g (0.116 mole) sodium hydroxide solution (19.7 wt. % in water). This solution was cooled in an ice water bath. After the solutions in both beakers had cooled, the sodium hydroxide solution was added in one shot to the cooled 3-chloro-2-hydroxypropyltrimethyl-ammonium chloride solution. The resulting mixture was then added in one shot to the starch slurry. The pH of the starch slurry was measured and found to be 10.79 at 43° C. An additional 16.5 mL of (0.0125 mole, 0.5 g) of 3.03% (0.76 N) NaOH was added to the slurry at a rate of 7.5 mL/minute using an automated dispenser. The pH of the slurry was measured and found to be 10.96 at 43° C.

The reaction was allowed to stir at 43° C. for 23 h. After 23 h. at 43° C. the pH of the slurry was 10.7. The reaction was cooled and neutralized with dilute HCl. The final pH was 5.51 at 22° C.

The starch was isolated by vacuum filtration and then the filter cake was washed with 5×400 mL deionized water on the funnel allowing the water to nearly entirely disappear into the filter cake between each wash (great care was taken to keep the filter cake from cracking). The dried product thus obtained was ground to a powder using a coffee grinder. The yield of dried product was 302.3 g (86.63% % solids by moisture balance).

### Jet-Cooking Modified Starch

In a 2 L beaker, 75.03 g of the starch prepared in Step B (65.00 g 100% basis starch) was added in one portion to 647.2 g deionized water, and the resulting mixture was stirred until a smooth slurry with no visible agglomerated particles was obtained. The slurry was then jet-cooked at 220-230° F. using the laboratory mini-jet-cooker with a flow rate through the cooker of about 125-130 mL/min.

The cook came out gelatinous and apparently homogenous. The entire cook was freeze-dried using a bulk tray freeze dryer. Yield of white solid: 66.3 g. Solids by moisture balance: 90.80%.

The amount of 3-trimethylammonium-2-hydroxypropyl starch ether was found to be 5.18 mole % (vs. starch AGU) by Carbon-13 NMR (measured on enzyme degraded product).

Introduction of Cationic Functionality by Reaction with 2,3-epoxypropyltrimethyl-ammonium chloride

A 1 L jacketed beaker was equipped with a mechanical stirrer and thermometer. To the beaker were charged 341.28 g deionized water and 50.08 g anhydrous sodium sulfate. After the sodium sulfate had completely dissolved, 290.71 g (1.5433 mole AGU) of cross-linked starch (99 ppm epichlorohydrin cross-linking; prepared as described above) was charged to the reaction mixture. To the resulting slurry was added 66.668 mL (0.0500 mole, 2.0 g) of 3% (0.75 N) NaOH at a rate of 7.5 mL/minute using an automated dispenser. The pH of the reaction mixture was measured at this point and found to be 11.41 at 25° C. The temperature of the reaction mixture was then raised to 43° C. by circulating warm fluid (44° C.) through the beaker jacket. After about 0.5 hour at this temperature, the pH of the reaction mixture was measured and found to be 11.06 (pH electrode set for measurement at 43° C.).

To the resulting mixture, was added 24.9807 g (0.1158 mole) of 2,3-epoxypropyltrimethylammonium chloride (QUAB® 151 from QUAB Chemicals; 70.28% active material in water) in one portion. The pH of the reaction mixture dropped to 11.03. Five (5) mL of 0.75 N NaOH was added to bring the pH to 11.07. The jacketed beaker was then loosely capped with a nylon cover, and the reaction was allowed to stir for 18.5 at 43° C.

After 18.5 h stirring at 43° C., the pH of the reaction had dropped to 10.93. Some water had evaporated overnight leaving a small ring of starch in the beaker above the water line. This was rinsed back into the reaction vessel with deionized water.

The reaction was allowed to cool to 20° C. The pH of the reaction was then brought down to 5.7 by the drop-wise addition of dilute HCl with stirring at 20° C.

The starch was isolated by vacuum filtration and then the filter cake was washed with 5×400 mL deionized water on the funnel allowing the water to nearly entirely disappear into the filter cake between each wash (great care was taken to keep the filter cake from cracking). The dried product thus obtained was ground to a powder using a coffee grinder. The yield of dried product was 276.7 g (93.17% solids by moisture balance).

The %N of the starch was 0.354% N (average of two values measured on an ELEMENTAR Vario MAX CN nitrogen analyzer. The % N value corrected for solids was 0.380% N.

The amount of 3-trimethylammonium-2-hydroxypropyl starch ether was found to be 4.17 mole % (vs. starch AGU) by Carbon-13 NMR (measured on enzyme degraded jet-cooked product).

Introduction of Hydrophobic Functionality to a Cross-Linked Cationic Starch by Reaction with 2-octen-1-yl succinic anhydride (OSA)

In a 600 mL beaker equipped with an overhead mechanical mixer and a thermometer, 97.85 g (93.75% solids) of a cationic cross-linked starch (3.20 mole % 3-trimethylammonium-2-hydroxypropyl starch ether; 50 ppm epichlorohydrin) was slurried in 230.94 g deionized water. The pH of the slurry was brought to 8.49 by the slow addition (<7.0 mL/minute) of 2.23 mL (0.0636 g, 0.00159 mole) 2.84% (0.712 N) NaOH. The reaction mixture was adjusted to 22-24° C. by immersion of the beaker in a water bath, and drop-wise addition via syringe of 1.6292 g (0.00775 mole) OSA over a period of 10 minutes was commenced. After the OSA addition was complete, the reaction was allowed to stir at 22-24° C. for 20 h while the pH of the reaction mixture was maintained at 8.4 by the controlled addition of 2.85% NaOH solution (Metrohm 718 STAT Titrino used in STAT mode). After 20 h, a total of 8.40 mL of 2.845 NaOH had been added. The reaction was stopped by adjusting the pH to 6.52 at 22° C., and the product was isolated by vacuum filtration. The filter cake was washed with 4×250 mL deionized water on the funnel allowing the water to nearly entirely disappear into the filter cake between each wash (great care was taken to keep the filter cake from cracking). The product was then air dried to constant weight. The dried product thus obtained was ground to a powder using a coffee grinder. The yield of dried starch was 98.0 g (93.86% solids by moisture balance). The level of OSA bound to the starch was found to be 1.59 wt. % by HPLC analysis; this corresponds to 1.24 mole % (vs. Starch AGU).

Preparation of Inclusion Complex of Starch Product with octadecyltrimethylammonium chloride (ODAC)

In a 2 L beaker, 4.4384 g (0.01275 mole) ODAC was dissolved in water. To the solution was added in one portion 75.83 g (64.99 g 100% basis, 0.4012 mole AGU) of a cross-linked starch (49 ppm epichlorohydrin). The resulting mixture was stirred until a smooth slurry with no visible agglomerated particles was obtained. The slurry was then jet-cooked using the laboratory mini-jet-cooker following the procedure described above.

The yield of freeze-dried product was 63.83 g. Solids by moisture balance: 93.49%.

### Hydroxypropylation of Cross-Linked Waxy Maize

Into a 1 L beaker equipped with a magnetic stir bar were added 50.0 g of anhydrous sodium sulfate and 375.0 g deionized water. After the sodium sulfate had completely dissolved 290.83 g (85.96% solids) of cross-linked starch (25 ppm epichlorohydrin) was added with stirring. To the resulting slurry was added 123.34 mL (0.0937 mole, 3.748 g) 3.04% (0.760 N) NaOH at a rate of 7.0 mL/minute using an automated dispenser. After the NaOH addition, the pH was 11.77 at 24° C.

The slurry was then transferred to a 1L Nalgene® bottle. To the slurry was then added 20.00 g (0.344 mole) of propylene oxide. The bottle was capped and placed in a heated (hot air) tumbler reactor set at 40° C. and allowed to tumble for 24 h. at 40° C. After 24 h at 40° C., the heater was turned off, and the reaction bottle was allowed to cool to room temperature while continuing to tumble.

The bottle was removed from the tumbler, and the reaction was allowed to cool to room temperature. The pH of the reaction mixture was then reduced to 3.0 from 12.0 at 23° C. by the drop-wise addition of ˜10% H₂SO₄ and the reaction was stirred at this pH for 1 h. The pH was then increased to 5.6 by the addition of 3.0% NaOH. The starch product was isolated by vacuum filtration, and then the filter cake was washed with 4×400 mL deionized water on the funnel allowing the water to nearly entirely disappear into the filter cake between each wash (great care was taken to keep the filter cake from cracking). The product was then air dried to constant weight. The dried product thus obtained was ground to a powder using a coffee grinder. The yield of dried starch was 304.6 g (86.21%% solids by moisture balance).

The amount of hydroxypropyl starch ether was found to be 13.0 mole % (vs. moles starch AGU) by Carbon-13 NMR (measured on enzyme degraded jet-cooked product).

Preparation of Waxy Maize Cross-Linked and Modified with 3-chloro-2-sulfopropionic acid (CSPA)

To a 1 L beaker equipped with an overhead mechanical stirrer and thermometer were charged 315.0 g deionized water, 209.30 g (86.00% solids) of cross-linked starch (99 ppm epichlorohydrin), and 60.0 g anhydrous sodium sulfate. To the resulting starch slurry was added 46.36 mL (0.0360 mole, 1.44 g) 3.11% (0.777 N) NaOH at a rate of 5 mL/minute using an automated dispenser. The pH of the slurry after the caustic addition was measured and found to be 10.97.

In a separate 100 mL beaker, 9.42 g (0.0476 mole) 3-chloro-2-sulfopropionic acid (prepared according to Tessler, M. M. U.S. Pat. Nos. 4,119,487, 1978; 95.3% active; 11.57 meq/g acidity) was dissolved in 10.2 g deionized water. The resulting solution was cooled in an ice/water bath, and then 25.20 g (0.130 mole) of 20.63 wt. % NaOH in water was added to the cooled solution. The pH of the neutralized CSPA solution was measured and found to be 8.0 at 22° C.

The CSPA solution was then added rapidly to the starch slurry with agitation. The pH of the reaction mixture was 10.5 after the CSPA addition. The reaction mixture was then heated to 40° C. using a warm water bath; the pH of the slurry at 40° was 10.2. The pH of the reaction was adjusted to 10.9 and then maintained at this pH by the controlled addition of 3.11% NaOH in water (Metrohm 718 STAT Titrino used in STAT mode) at 40° C. for 21 h. After 21 h, a total of 52.75 mL 3.11% (0.777 N) NaOH had been added. The reaction was then cooled to ambient temperature, and the pH was adjusted to 5.9 by the addition of dilute HCl.

The product was isolated by vacuum filtration and then washed with 3×600 mL deionized water on the funnel allowing the water to nearly entirely disappear into the filter cake between each wash (great care was taken to keep the filter cake from cracking). The product was then air dried to constant weight. The dried product thus obtained was ground to a powder using a coffee grinder. The yield of dried starch was 206.2 g (88.55% solids by moisture balance).

The amount of 2-sulfo-2-carboxyethyl starch ether was found to be 1.95 mole % (vs. starch AGU) by Carbon-13 NMR (measured on enzyme degraded product).

Preparation of Waxy Maize Cross-Linked and Modified with 2-chloroethylaminodipropionic acid (CEPA)

Waxy maize starch, 224.16 g (89.22% solids), was cross-linked with 50 ppm epichlorohydrin and then modified with CEPA according to the method of Bernard, K. A.; Tsai, J.; Billmers, R. L.; Sweger, R. W. U.S. Pat. No. 5,455,340, 1995. The CEPA treatment level was 4.71 mole % (vs. starch AGU). The yield of dried starch was 228.8 g (89.84% solids by moisture balance).

The amount of (2-aminodipropionic acid)ethyl starch ether was found to be 2.04 mole % (vs. starch AGU) by Carbon-13 NMR (measured on enzyme degraded product).

### Emulsion Preparation

A) No Added Co-surfactant

A 400 mL beaker equipped with mechanical stirring was charged with water (93.5 g). The stirring was set to slow and then the beaker was slowly charged with the experimental polymer (1 g) by sifting into the vortex. The solution was allowed to mix until homogenous. At this point the agitation was increased to 900-1200 rpm and the oil (tetradecane, 5 g) was added to the mixture by the dropperful. The mixture was allowed to mix for 30 minutes. At this point a preservative (GLYDANT®, 0.5 g) was added. The emulsion was allowed to sit overnight before beginning evaluations.

B) Added Co-Surfactant

A 400 mL beaker equipped with mechanical stirring was charged with water (93.4 g). The stirring was set to slow and then the beaker was slowly charged with the experimental polymer (1 g) by sifting into the vortex. The solution was allowed to mix until homogenous. At this point the agitation was increased to 900-1200 rpm and the oil (a premixed solution of tetradecane (5 g) and Span 80 (0.1 g)) was added to the mixture by the dropperful. The mixture was allowed to mix for 30 minutes. At this point a preservative (GLYDANT®, 0.5 g) was added. The emulsion was allowed to sit overnight before beginning evaluations.

### Traditional Stability

The emulsion is evaluated before aging for viscosity (Brookfield Heliopath RVT viscometer using spindle C at 10 rpm using at least 6 revolutions and the correct factor to determine viscosity), pH, subjective evaluations for shine, flow, texture, color, and clarity, and examined through a 250× brightfield microscope. The microscope is used to estimate the average droplet size (averaging 10 drops and using the micrometer equipped ocular), checking for the absence of polar birefringence using the polarized filters, and taking digital pictures if appropriate.

One sample is aged under ambient laboratory conditions, one in storage at 45° C., and one sample frozen and then thawed. The jars are checked frequently so that if the emulsion fails the time to a phase separation can be noted. After four weeks the samples that underwent ambient or 45° C. aging are characterized with the same tests as conducted initially. The batch is considered to pass if the emulsion does not phase over the full four weeks at 45° C.

### Effect of Cross-Linking Concentration on Emulsion Stability

Samples were prepared using the procedure above and were treated with various amounts of epichlorohydrin ranging from 0 to 400 ppm treatment based on the starch weight. All samples had approximately the same quaternary nitrogen content and were analyzed for emulsion stability using 1% starch (as is based on the total formulation weight) and 5% tetradecane as the oil.

TABLE 1
Effect of cross-linking concentration and type

| Sample | crosslink | | starch | cationic reagent | | |
|---|---|---|---|---|---|---|
| # | ppm | type | base | mole % | type | STABILILTY |
| 1 | 400 | epi | waxy | 4.68 | QUAB ® 188 | FAIL |
| 2 | 200 | epi | waxy | 5.18 | QUAB ® 188 | PASS |
| 3 | 100 | epi | waxy | 4.17 | QUAB ® 188 | PASS |
| 4 | 50 | epi | waxy | 3.20 | QUAB ® 188 | PASS |
| 5 | 25 | epi | waxy | 4.90 | QUAB ® 188 | PASS |
| 6 | 10 | epi | waxy | 4.78 | QUAB ® 188 | FAIL |
| 7 | 0 | | waxy | 4.53 | QUAB ® 188 | FAIL |
| 18 | 50 | epi | waxy | 2.59 | QUAB ® 188 | PASS |
| | | | rice | | | |

Table 1 shows that a minimum level of cross-linking reagent of about 10 ppm or greater is needed to achieve acceptable emulsion stability. Additionally there is a maximum of about 400 ppm of cross-linking reagent, above which the starch is no longer effective in providing a stable emulsion.

### Cross-Linked Anionic Starches Evaluated for Emulsion Stability

Anionic and non-ionic starches were prepared as described above with similar levels of cross-linking. Anionic materials were CSPA, CEPA and OSA modified starches. Under conditions of the emulsification, the acid groups tend to be neutralized All samples were tested with and without additional surfactant (SPAN 80), which when present in added at 0.1% use level as described in the testing section). The surfactant alone did not provide any emulsion stability when tested at 0.1%. In addition, the surfactant alone did not provide emulsion stability even when tested at higher levels.

TABLE 2
Emulsifying properties of modified starches
with and without surfactant.

| | | | | Modifying | | STABILITY | |
|---|---|---|---|---|---|---|---|
| Sample | crosslink | | starch | reagent | | w/o | W/surfac- |
| # | ppm | type | base | mole % | type | surfactant | tant |
| 11 | 100 | epi | waxy | 1.95 | CSPA | FAIL | PASS |
| 12 | 50 | epi | waxy | 2.12 | CSPA | FAIL | PASS |
| 13 | 50 | epi | waxy | 2.63 | CEPA | FAIL | PASS |
| 14 | 50 | epi | waxy | 2.04 | CEPA | FAIL | PASS |
| 10 | 50 | epi | waxy | 13.16 | PO | FAIL | FAIL |
| 16 | 50 | epi | waxy | 6.00 | OSA | FAIL | FAIL |
| 17 | 0 | | waxy | 2.89 | OSA | FAIL | FAIL |
| Ref 1 | — | — | — | — | — | FAIL | FAIL |
| Ref 2 | — | — | — | — | — | FAIL | FAIL |

The results in table 2 demonstrate that CEPA and CSPA starches are capable of making acceptable emulsions in the presence of small amount of the non-ionic surfactant. In the absence of the starch (sample Ref 1 is at 0.1% Span 80 and Ref 2 is 5.0% span 80) no stable emulsion could be formed. It should be noted that neither cross-linked nor un-cross-linked OSA modified waxy starch (samples 16 and 17) provide the required emulsion stability, which would have been expected based on the teachings of the prior art. Also the hydroxypropyl (PO) starch (sample 10) did not form a passing emulsion with or without the addition of the surfactant.

Emulsions Made with Various Quaternary Nitrogen Concentrations

Samples shown in table 3 were prepared by the methods described above with similar cross-linking levels but with varying amounts of quaternary reagent. In this example samples containing covalently bound and complexed quats were prepared and tested. These emulsions were prepared using only the modified starch, oil, and water. No surfactant needed to be added for these samples.

TABLE 3
Effect of cationic substituent

| Sample | Crosslink | | starch | cationic reagent | | Hydrophobic Cationic | | |
|---|---|---|---|---|---|---|---|---|
| # | ppm | Type | base | mole % | type | mole % | Type | STABILITY |
| 8 | 50 | epi | waxy | 0.00 | QUAB ® 188 | | | FAIL |
| 15 | 25 | epi | waxy | 1.00 | QUAB ® 188 | | | FAIL |
| 4 | 50 | epi | waxy | 3.20 | QUAB ® 188 | | | PASS |
| 18 | 50 | epi | waxy | 3.20 | ODAC | | | PASS |
| | | | rice | | | | | |
| 19 | 50 | epi | waxy | 3.20 | ODAC | | | PASS |
| 20 | 50 | epi | waxy | 3.12 | QUAB ® 188 | 1.51 | QUAB ® 342 | PASS |

The results above show that greater than about 1 mole percent of cationic reagent must be attached to the starch to achieve emulsion stability. Sample 4 demonstrates the use of a bound cationic reagent and Sample 19 demonstrates the use of a complexed cationic reagent to form stable emulsion. Sample 20 demonstrates the compatibility of short and long chain cationic reagents to form effective polymeric emulsifiers.).

Although the invention is illustrated and described herein with reference to specific embodiments, the invention is not intended to be limited to the details shown.

Rather, various modifications may be made in the details within the scope and range of equivalents of the claims and without departing from the spirit of the invention.

What is claimed:

1. A polymeric emulsifier comprising a polysaccharide modified with at least one cross-linking reagent and with from about 1 mol % to about 10 mol % of at least one short chain cationic reagent, wherein the modified polysaccharide is further modified with at least about 15 ppm and less than 100 ppm of the cross-linking reagent.

2. The polymeric emulsifier of claim 1 wherein the polysaccharide is starch.

3. The emulsifier of claim 2 wherein the polysaccharide is gelatinized starch.

4. The emulsifier of claim 2 wherein the starch contains about 95 percent or more of amylopectin based on the total weight of the starch.

5. The emulsifier of claim 1 wherein the modified polysaccharide is further modified with from about 25 ppm to about 75 ppm of the cross-linking reagent.

6. The emulsifier of claim 1 wherein the at least one cationic reagent comprises 3-chloro-2-hydroxypropyltrimethyl ammonium chloride or 2,3 epoxypropyltrimethyl ammonium chloride.

7. The emulsifier of claim 1 wherein the starch is modified with from about 1 to about 5 mole percent of a long chain cationic reagent having the Structure I: 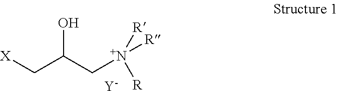
wherein R′ and R″ are alkyl groups having one to three carbon atoms and R is an alkyl group with from about 8 to about 18 carbon atoms; X and Y each are chlorine.

8. A polymeric emulsifier comprising a polysaccharide modified with at least one cross-linking reagent and with from about 1 mol % to about 10 mol % of at least one anionic containing reagent, wherein the polysaccharide is modified with from about 15 ppm to about 375 ppm of the cross-linking reagent.

9. The emulsifier of claim 8 wherein the at least one anionic containing reagent is 3-chloro-2-sulfopropionic acid or 2-chloroethylaminodipropionic acid.

10. The emulsifier of claim 8 wherein the emulsifier further comprises a non-ionic surfactant.

11. An emulsion comprising:
a polymeric emulsifier comprising a polysaccharide modified with at least one cross-linking reagent and at least one ionic reagent;
a cosmetically acceptable oil; and
a solvent system,
wherein the solvent system is the continuous phase.

12. The emulsion of claim 11 wherein the polysaccharide is modified with from 1 mol % to 10 mol % of said ionic reagent.

13. The emulsion of claim 11 wherein the ionic reagent comprises at least one short chain cationic reagent and wherein the modified polysaccharide is further modified with at least about 15 ppm and less than 100 ppm of the cross-linking reagent.

14. The emulsion of claim 11 wherein the polysaccharide is starch.

15. The emulsion of claim 11 wherein the polysaccharide is gelatinized starch.

16. The emulsion of claim 11 wherein the starch contains about 95 percent or more of amylopectin based on the total weight of the starch.

17. The emulsion of claim 11 wherein the modified polysaccharide is modified with from about 25 ppm to about 75 ppm of the cross-linking reagent.

18. The emulsion of claim 13, wherein the at least one short chain cationic reagent comprises 3-chloro-2-hydroxypropyltrimethyl ammonium chloride or 2,3 epoxypropyltrimethyl ammonium chloride.

19. The emulsion of claim 11 wherein the starch is further modified with from about 1 to about 5 mole percent of a long chain cationic reagent having the Structure I: 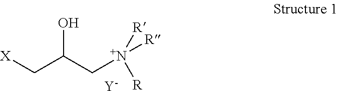
wherein R′ and R″ are alkyl groups having one to three carbon atoms and R is an alkyl group with from about 8 to about 18 carbon atoms; X and Y each are chlorine.

20. The emulsion of claim 11 wherein the ionic reagent is at least one anionic containing reagent and wherein the polysaccharide is modified with from about 15 ppm to about 375 ppm of the cross-linking reagent.

21. The emulsion of claim 20 wherein the at least one anionic containing reagent is 3-chloro-2-sulfopropionic acid or 2-chloroethylaminodipropionic acid.

22. The emulsion of claim 21 wherein the emulsifier further comprises a non-ionic surfactant.

23. The emulsion of claim 11 wherein the solvent system is a mixture of water and one or more of ethanol, methanol, isopropanol, glycerol, propylene glycol or acetone.

24. A personal care formulation comprising the emulsion of claim 11 wherein the formulation is a hair mousse, styling gel, sunscreen, hair conditioner or moisturizer.

25. The emulsion of claim 11 wherein the cosmetically acceptable oil is an aerosol propellant.
